(19) United States
(12) Patent Application Publication
(10) Pub. No.: US 2026/0237503 A1
(43) Pub. Date: Aug. 13, 2026

(54) SYSTEMS AND METHODS FOR RECOMMENDING UPGRADES FOR A FLEET OR INVENTORY OF MEDICAL DEVICES

(71) Applicant: KONINKLIJKE PHILIPS N.V., EINDHOVEN (NL)

(72) Inventors: Sauvik Bhattacharya, EINDHOVEN (NL); Johannes Henricus Maria Korst, EINDHOVEN (NL); Andrei Poliakov, EINDHOVEN (NL); Roman Nikitchenko, EINDHOVEN (NL); Falk Uhlemann, NORDERSTEDT (DE); Robert Paul Koster, EINDHOVEN (NL); Ramon Antoine Wiro Clout, EINDHOVEN (NL)

(21) Appl. No.: 19/159,653

(22) PCT Filed: Feb. 20, 2024
(86) PCT No.: PCT/EP2024/054212

§ 371 Date: Aug. 26, 2025

Related U.S. Application Data
(60) Provisional application No. 63/450,469, filed on Mar. 7, 2023.

(51) Int. Cl. G16H 40/40

(52) U.S. Cl. CPC G16H 40/40

(57) ABSTRACT

Data related to a plurality of medical devices and potential upgrades to the plurality of medical devices is stored. In an upgrade recommender method, a plurality of different potential upgrades are determined for the plurality of medical devices. A set of customer priorities and/or requirements are received. A score is assigned to each potential upgrade based on the received set of customer priorities and/or requirements, and an indication is output of at least the highest-scoring potential upgrade. A compatibility status between multiple medical devices of the plurality of medical devices may also be determined from the data related to the plurality of medical devices, and for the different potential upgrades, and these compatibility statuses displayed.

### FIELD

The following relates generally to the medical device maintenance arts, medical device inventory maintenance arts, medical device upgrade arts, and related arts.

### BACKGROUND

Connected medical solutions are modular systems, consisting of interoperating, independent devices. An example is a catheterization laboratory consisting of an interventional suite including an X-ray generator, image intensifier, viewing monitors, and operating work spot. Another example is a patient monitoring system consisting of bedside monitors connecting with various sensor devices (temperature sensors, blood pressure monitors, SpO₂ sensors, and/or so forth) and interoperating with a central nurse workstation. Full functionality of such a modular medical system relies on reliable interconnections between the constituent systems, subsystems, or components. Functionality can be lost if, for example, a patient monitor is unable to operatively connect with a feature of a given sensor. As just one example, if a patient sensor measures both pulse rate and SpO₂ level but its connection with a patient monitor conveys only the SpO₂ data, then the pulse rate measurement functionality is lost. Full functionality can also be adversely impacted if the hospital has multiple models of a component in stock, only some of which are interconnectable with full functionality with other components. To take the immediately preceding example, if the hospital has two different models of patient monitor in stock, but only one model connects with the patient sensor with full SpO₂ and pulse rate functionality, then problems can arise as the sensor will only work with full functionality when connected with the fully compatible model of patient monitor. This type of inventory compatibility complexity increases exponentially with larger hospitals having larger inventories, often accumulated over multiple iterations of equipment purchases and/or leases.

Upgrading such composite systems or inventories is more involved compared to upgrading a traditional single medical device, such as software on a Magnetic Resonance system. The updated version of a component and the current versions of its connected devices should be compatible. Furthermore, upgrading all components to their latest respective versions is not always necessary nor the most cost-effective for the user (i.e., a hospital). “Cost” here can refer to monetary cost, or the time required for the upgrade during which the system is not operational.

In addition, hospitals have diverse information technology (IT) environments, policies and requirements. Frequently a hospital consists of multiple clinical departments, each with a set of diverse medical equipment from different manufacturers, with different functions, versions, and networking requirements. The hospital can also have diverse networking and security policies in place for different departments, and each can have their own requirements for functionality and cost of equipment. Thus, hospitals are increasingly becoming a heterogeneous environment into which medical systems must be integrated, and various functionality, security and cost requirements need to be accounted for during such integration.

Medical systems are also increasingly composed of multiple interacting modules. Upgrading such systems can happen in one monolithic step by upgrading all components to the available next higher version, or various permutations based on the kind of customer needs. Hospital staff can be disappointed to find that newly acquired medical devices are not fully compatible with, or wholly incompatible with, existing inventory of medical devices with which the new devices were intended to co-function.

The following discloses certain improvements to overcome these problems and others.

### SUMMARY

In one aspect, a non-transitory computer readable medium stores data related to a plurality of medical devices, and instructions readable and executable by at least one processor to: determine, from the data related to the plurality of medical devices, a compatibility status between multiple medical devices of the plurality of medical devices; and output, on a graphical user interface (GUI) displayed on a display device, an indication of the compatibility status. The data related to a plurality of medical devices may, for example, comprise a representation including (i) the medical devices, (ii) connections therebetween, and (iii) formulations for checking for incompatibilities between the medical devices. In some nonlimiting illustrative examples, the instructions are further executable by the at least one processor to: determine a compatibility status of a potential software upgrade of the plurality of medical device by performing the determine and output operations for the plurality of medical devices with the potential upgrade to output, on the GUI, an upgrade compatibility status for the potential upgrade; and receive, via the GUI, an instruction to perform the upgrade and in response automatically push the software upgrade to the plurality of medical devices over an electronic network.

In another aspect, a non-transitory computer readable medium stores data related to a plurality of medical devices and potential upgrades to the plurality of medical devices, and instructions readable and executable by at least one processor to: receive or determine a plurality of different potential upgrades for the plurality of medical devices; provide a GUI displayed on a display device via which a set of customer priorities and/or requirements are received; assign a score to each potential upgrade based on the received set of customer priorities and/or requirements; and output, on the GUI, an indication of at least the highest-scoring potential upgrade. In some embodiments, the received set of customer priorities and/or requirements includes performance requirements for the medical devices including at least one of sharpness of images captured by the medical devices, resolution of images captured by the medical devices, speed of operation of the medical devices, and data communication latency of the medical devices. In some nonlimiting illustrative examples, the instructions are further readable and executable by the at least one processor to receive, via the GUI, a selection of a potential software upgrade indicated by the output for implementation, and in response to the selection, automatically push the software upgrade to the plurality of medical devices over an electronic network.

In another aspect, a non-transitory computer readable medium stores data related to a plurality of medical devices, and instructions readable and executable by at least one processor to: determine, from the data related to the plurality of medical devices, a compatibility status between multiple medical devices of the plurality of medical devices, the compatibility status between the multiple medical devices of the plurality of medical devices is a compatibility of an operative connection between a pair of medical devices of the plurality of medical devices; and output, on a GUI displayed on a display device, an indication of the compatibility status.

One advantage resides in providing continuous monitoring of potential incompatibilities and automatic calculation of possible update scenarios for upgrading medical devices.

Another advantage resides in providing manually or automatically triggered updates for medical devices, thereby increasing security of the medical device during the update.

Another advantage resides in providing increased efficiency of medical device operation based on an update.

Another advantage resides in maximizing compatibility by maximizing interconnectivity of newly acquired medical devices with the existing inventory of medical devices.

Another advantage resides in increased customer workflow execution efficiency based on tailored updates for medical devices.

Another advantage resides in proactively sharing actionable information on the various options possible for performing updates of medical devices.

Another advantage resides in only updating medical devices in a hospital when resulting improvements to the devices because of the update outweigh security and functionality concerns.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for updating medical devices in accordance with the present disclosure.

FIG. 2 shows exemplary flow chart operations of the system of FIG. 1.

FIG. 3 diagrammatically illustrates a chart generated by the operations of FIG. 2.

FIGS. 4A and 4B shows another embodiment of the system of FIG. 1, implemented as a client-server system where FIG. 4A shows the client side and FIG. 4B shows the server side.

### DETAILED DESCRIPTION

The following relates to upgrading/updating interacting medical devices. In one example embodiment, a hospital-wide patient monitoring system may include individual monitoring modules (e.g. vital sign sensors for various vital signs, mechanical ventilators that also monitor patient respiration, etc.) that are connectable with multifunction patient monitors, which in turn are connectable with an information center (e.g., at a nurses' station). In this embodiment, the monitoring modules are a device type which is connectable with the multifunction patient monitor device type but not with the information center device type. Moreover, a given monitoring module might not be connectable with a given multifunction patient monitor if, for example, they employ incompatible connectors or connection protocols (e.g. USB versus serial ports, or Bluetooth versus Wi-Fi).

In some embodiments, a system for checking upgrades for incompatibilities that could degrade or break the overall system is disclosed. An upgrade can in general be a software upgrade or a hardware upgrade or a combination thereof (e.g. switching the hospital IT network from wired Ethernet to Wi-Fi which would involve both hardware and software updates). The disclosed system provides a detailed directed graph-based mathematical representation of the medical devices and connections therebetween, and formulations for checking for incompatibilities. In some embodiments incompatibility is a binary output (compatible or incompatible). In other embodiments, the incompatibility can be a probabilistic output, for example to represent a device connection that is mostly compatible, but which may break some non-medical functionality that would not impact patient safety, such as a module collecting information on network connectivity reliability.

This disclosed system may be a primarily vendor-facing user interface (UI), though some components such as provision for “what-if” scenario analyses could be customer-facing to enable the customer to directly investigate possible upgrades.

In other embodiments, a system provides a customer-facing UI that enables customers to run “what-if” scenarios on possible upgrades/updates that take into account various customer interests (e.g. monetary cost, system downtime during upgrade, compatibility of upgraded devices with existing devices, etc.) in providing personalized upgrade path recommendations. The user inputs the customer interests, which are converted to weights and input to a scoring module. The incompatibility checker is applied to determine any incompatibilities, and a list of top-N personalized upgrade paths are provided. The recommended upgrades may for example recommend upgrading only some patient monitors to limit the impact of downtime for the upgrade, or may recommend upgrading software to a version that is not the latest version to avoid incompatibilities, or so forth. The disclosed system provides a detailed mathematical formulation for assessing the various upgrade options to develop the personalized list of recommendations.

In addition the disclosed system is configured to retain the previous configuration (i.e. without the upgrades) as a recovery point. If the upgrade fails for any reason (for example, the new software being unable to work with the hospital IT infrastructure firewall), then the customer can recover back to that recovery point. This feature is typically most useful in the case of software upgrades, but could also be useful for hardware upgrades by tracking which purchased or leased medical devices should be returned and re-acquired to reverse an unsuccessful upgrade.

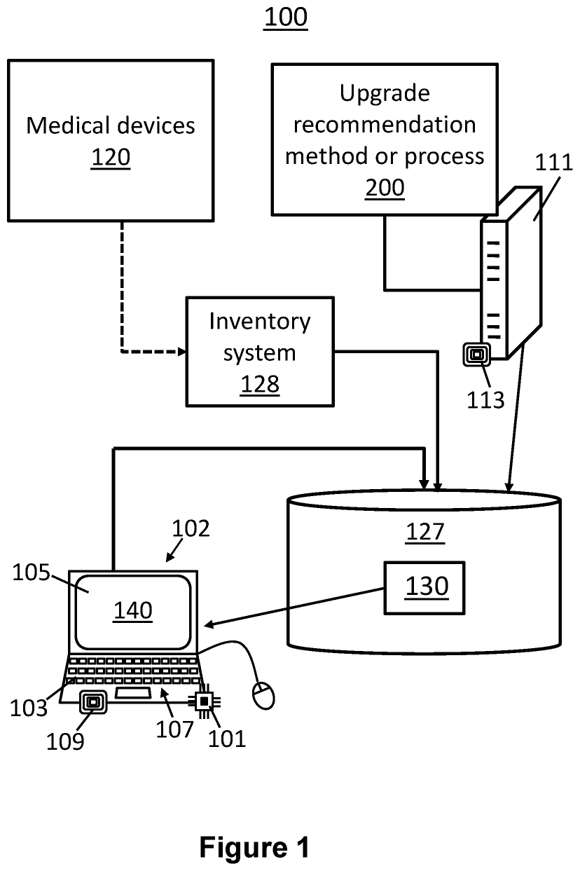

With reference to FIG. 1, an illustrative servicing support system 100 for supporting a service engineer in servicing a medical device or fleet or inventory of medical devices 120 is diagrammatically shown. By way of some non-limiting illustrative examples, the medical device(s) 120 subject to upgrade may be patient monitors, medical imaging devices (e.g., a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth), an inventory of mechanical ventilators, and/or so forth. (More generally, the disclosed approach can be applied in conjunction with any type of computerized device or machine that requires automated software updates, e.g., the approach could be applied to a commercial airliner, radiation therapy device, a cargo ship, an industrial robot, or so forth). It should be appreciated that in the setting of a large hospital or a large network of hospitals, the number of medical devices 120 being upgraded could be large, e.g. dozens or hundreds or more medical devices, possibly with multiple models of a given type of medical device such as multiple models of patient monitor, and possibly with medical devices of a given type purchased or leased possible from multiple different vendors. Still further, the medical devices 120 may have various types of interconnectivity, such as wired connections with specific connector types, wireless connectivity in accordance with specific wireless communication protocols, and/or so forth. Hence, upgrading the fleet or inventory of medical devices 120 while maintaining a high degree of (ideally complete) functional interconnectivity between the upgraded medical devices is challenging.

As shown in FIG. 1, the upgrade support system 100 includes, or is accessible by, a vendor device 102 that may for example be a workstation or electronic processing device. The vendor device 102 is operated by an employee or other agent of a vendor (for example, by a customer service representative, or so forth), and may for example be a desktop computer or a portable device such as a notebook computer. As another nonlimiting illustrative example, the vendor device 102 may be a mobile device such as a cellular telephone (cellphone) or tablet computer.

The vendor device 102 includes a display 105, at least one user input device 103 such a mouse, keyboard, or touchscreen. The vendor device 102 further includes an electronic processer 101 and non-transitory storage medium 107 (internal components which are diagrammatically indicated in FIG. 1). The non-transitory storage medium 107 stores instructions which are readable and executable by the electronic processor 101 for interfacing the vendor (e.g., customer service representative, or other agent of the vendor) with the support system 100.

The vendor device 102 also includes a communication interface 109 to communicate with a backend server or processing device 111, which implements the computational aspects of the support system 100. Such communication interfaces 109 include, for example, a wired and/or wireless Ethernet interface; or in the case in which the vendor device 102 is a portable FSE device the interface 109 may be a wireless Wi-Fi or 4G/5G interface or the like for connection to the Internet and/or an intranet. Some aspects of the support system 100 may also be implemented by cloud processing or other remote processing (that is, the server computer 111 may be embodied as a cloud-based computing resource comprising a plurality of interconnected servers).

The server 111 is equipped with non-transitory storage medium 127 (internal components which are diagrammatically indicated in FIG. 1). While a single server computer is shown, it will be appreciated that the server 111 may more generally be implemented on a single server computer, or a server cluster, or a cloud computing resource comprising ad hoc-interconnected server computers, or so forth. It will be appreciated that there may be multiple instances of the vendor device 102 enabling various customer representatives, and/or other vendor agents.

With continuing reference to FIG. 1, the non-transitory computer readable medium 127 stores data related to a plurality of medical devices 120. For examples, the stored data can include data on: (i) the medical devices 120, (ii) connections therebetween, and (iii) look-up tables and/or other formulations for checking for incompatibilities between the medical devices. Current data on the fleet or inventory of medical devices 120 may be retrieved, for example, from a hospital inventory system 128 that records information on the inventory of medical devices 120 for diverse purposes such as allocation of medical devices to hospital departments or laboratories, tracking of material assets for legal and tax purposes, and/or so forth. (In diagrammatic FIG. 1, the connection from the medical devices 120 to the inventory system 128 is diagrammatically indicated by a dashed arrow, to indicate that information on the medical devices 120 is typically indirectly entered into the inventory system 128 by human data entry operators or other indirect mechanisms).

The non-transitory storage medium 127 further stores instructions executable by the electronic processor 113 of the backend server 111 to perform an upgrade recommendation method 200 for recommending one or more potential updates of the medical devices 120. The potential updates can include, for example, a hardware update, a software update, or a combination thereof.

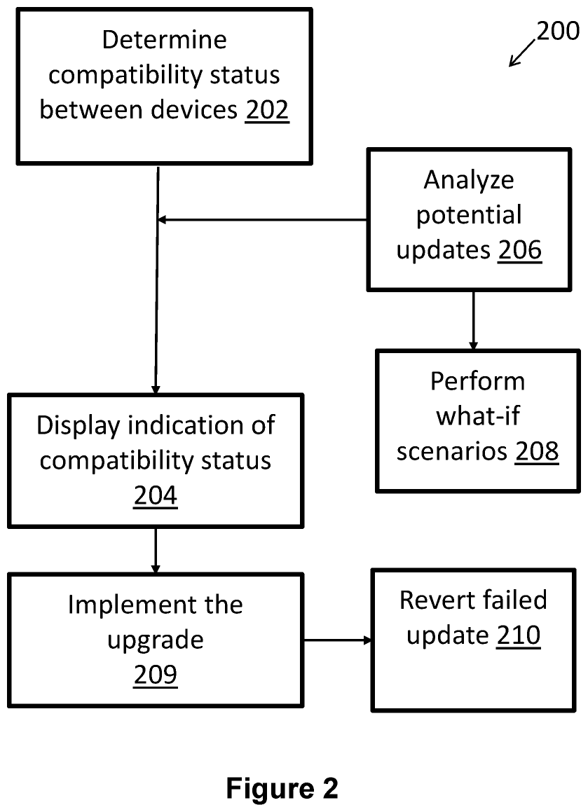

With continuing reference to FIG. 1 and further reference to FIG. 2, an illustrative embodiment of the upgrade recommendation method 200 is diagrammatically shown as a flowchart. In some examples, the method 200 may be performed at least in part by cloud processing. The upgrade recommendation method 200 in general operates to generate a recommended upgrade to the medical devices 120 based on information on those devices (e.g., from the inventory system 128) and their interconnectivity along with interconnectivity information for the contemplated devices of the one or more proposed upgrade paths. The upgrade recommendation method 200 proposes an upgrade based on factors such as maximizing interconnection compatibility with the existing inventory of medical devices 200 and various user-supplied requirements such as acceptable price range, number of new medical devices to be purchased or leased (in the case of a hardware upgrade), and potentially other factors such as delivery time, new device source (e.g., if the hospital has preferred source goals), et cetera.

To begin the upgrade recommendation method 200, at an operation 202, the data related to the medical device(s) 120 is retrieved, e.g. from the inventory system 128, and a compatibility status between multiple medical devices of the plurality of medical devices is determined. In one example, the compatibility status comprises a binary representation, (i.e., a “compatible” status or an “incompatible” status). In another example, the compatibility status comprises a probability of compatibility between multiple medical devices 120.

In one embodiment, the compatibility status between the multiple medical devices 120 is a compatibility of an operative connection between a pair of medical devices 120. For example, to determine the compatibility status, a connected graph 130 is generated. The connected graph 130 includes nodes representing the medical devices 120, and edges between pairs of nodes. Each edge represents an operative connection between the medical devices represented by the nodes connected by that edge. The edges are labeled with compatibilities of the operative connections between the medical devices 120 represented by the nodes connected by the respective edges. If there are n devices in the inventory of devices 120 then the number of pairs to be analyzed, and hence the number of possible edges in the graph 130, is

$$C ( n , 2 ) = ( \begin{matrix}n \\ 2\end{matrix} ) = \frac{n ( n - 1 )}{2}$$

As a specific example, if n=200 medical devices then there are

$$\frac{200 \times 199}{2} = 19 , 900$$

device pairs, to analyze, and hence 19,900 possible edges in the graph 130. This presenting a substantial challenge for assessing device interconnectivity and recommending possible upgrades.

In some examples, one or more new edges can be added to the connected graph 130, and the determination of the compatibility status includes determining compatibility statuses for the one or more new edges. In another example, data related to a proposed new medical device can be added to the data related to the plurality of medical devices 120, and the compatibility status is between the proposed new medical device and at least one other medical device 120 with which the proposed new medical device is proposed to be connected. These are merely illustrative examples, and should not be construed as limiting.

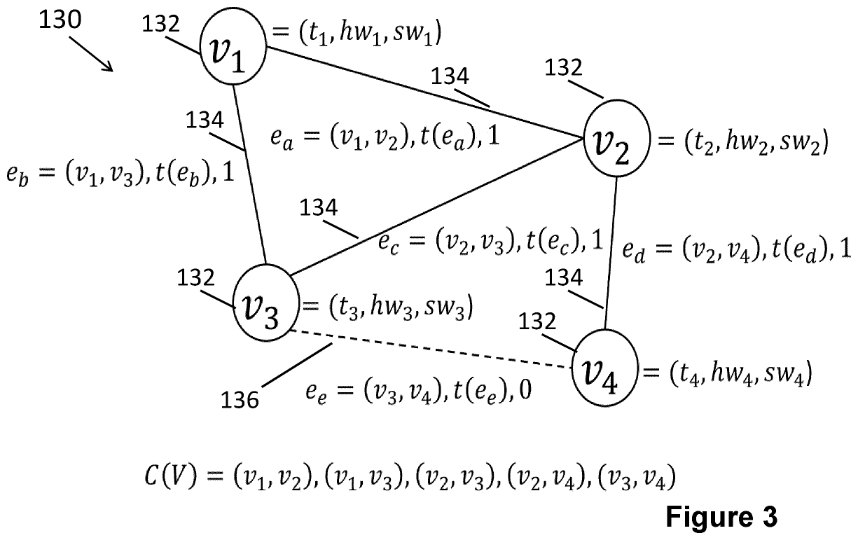

At an operation 204, an indication of compatibility status is displayed. In one approach, the connected graph 130 is displayed on a graphical user interface (GUI) 140 on the display device 105. FIG. 3 shows an example of the connected graph 130. The connected graph 130 is a representation of a network with four connectable (represented by set C (V)) devices as nodes 132 and four connections as edges 134. A binary flag for each connection between a pair of connectable devices represents whether that connection actually exists in a given network configuration. As shown in FIG. 3, the connection eₑ is possible but does not exist (indicated with a dashed line and reference character 136). In other embodiments, the operation 204 displays the indication of compatibility status in a more summary form, such as displaying a list of incompatible device pairs. For applications in which the inventory of devices 120 is heterogeneous, this summary list of incompatible device pairs may be filtered to remove “expected” incompatible pairs. For example, in a medical monitoring devices inventory, devices of the sensor type may be expected to connect with devices of the medical monitor type; but, devices of the sensor type may not be expected to connect with each other. Hence, incompatible sensor-sensor device pairs are “expected” incompatible pairs and thus would not be included in the list of incompatible device pairs, while any cases of incompatible sensor device-medical monitor pairs would be listed.

Referring back to FIG. 2, at an optional operation 206, potential updates for the plurality of medical devices 120 are analyzed, and the compatibility status is determined based on the analyzed potential updates. To do so, the determination of the compatibility status between multiple medical devices 120 is repeated for each of a plurality of different potential upgrades to the plurality of medical devices 120. A proposed upgrade can include replacing medical devices and/or adding new medical devices. Replacing a medical device amounts to replacing a node of the connected graph 130 representing the device to be replaced with a replacement node representing the proposed replacement device, and all edges connecting to that replacement node are then re-calculated. Adding a new device amounts to adding a new node to the connected graph 130, and all edges between that new node and each existing node are calculated. In another embodiment, the proposed upgrade could be a software update to the existing plurality of medical devices 120 which could impact the compatibility status. For example, the software upgrade could update the communication protocol used in wired or wireless communications in a way that might cause previously intercommunicating devices to be unable to communicate, e.g. if some older devices are unable to receive the software update then they may be unable to have their communication protocol updated and hence may be unable to communicate with other devices employing the updated communication protocol. A score is assigned to each potential upgrade based on the compatibility status for that potential upgrade (determined, for example, based on a sum or other aggregation of the pairwise compatibilities calculated for the new edges connecting with the replacement or new node) and a set of customer priorities and/or requirements. The connected graph 130 can optionally include indications of the scores assigned to the respective potential upgrades, or if the output 204 is a list of incompatible device pairs this list can be updated based on the compatibility values calculated for the new edges connecting with each replacement node or new node representing the upgrade. A user dialog via which the set of customer priorities and/or requirements is received is displayed on the GUI 140. Optionally, a list of potential upgrades and/or the scores based on the determined compatibility status can be displayed on the GUI 140. The output 204 for the potential update enables the user to decide whether the potential update is acceptable given its impact on compatibility status of the plurality of medical devices 120. If so, then in an operation 209 the user provides an instruction via the GUI 140 to perform the upgrade. In the case of a hardware upgrade, this may bring up a service scheduler to enable the user to schedule a technician or other qualified personnel to perform the hardware upgrade. In the case of a software upgrade that can be pushed automatically to the plurality of medical devices 120 via the Internet or another suitable electronic network, the user instruction to perform the upgrade can cause the operation 209 to automatically push the software update to the plurality of medical devices 120 over the Internet or other electronic network.

At an optional operation 208, an input indicative of a selection of one or more of the determined potential updates is received via the GUI 140. For example, the plurality of different potential upgrades for the plurality of medical devices 120 are received via the GUI 140 as what-if scenarios for upgrading the plurality of medical devices. The what-if scenarios are performed on the data related to the medical devices 120 and potential upgrades to the medical devices 120. The what-if scenarios can include analyzing one or more customer factors for the potential upgrades, including one or more of monetary cost, downtime, and compatibility of updated medical devices with non-updated medical devices, and selecting one or more potential upgrades based on the analyzing. The potential upgrades are then scored based on the analyzing of the one or more customer factors.

At an optional operation 210, a status of each medical device 120 prior to any implemented update is stored in the non-transitory storage medium 127 and, responsive to a failed update, reverting the “failed” medical devices to the stored status.

In the following, some nonlimiting illustrative examples are provided to illustrate further aspects which are to be understood as being amenable to being variously combined, with certain aspects optionally being omitted in certain specific implementations.

### First Example

For connected, multi-device medical solutions where individual devices need upgrading, the disclosed system 100 avoids breaks in functionality due to incompatibility between components and increases efficiency by computing, visualizing and allowing the user to choose between multiple upgrade paths towards a configuration with no incompatibilities. Each upgrade path has its own cost and benefit, and thus value to the user depending on their needs. If numerically quantifiable, these needs can be integrated into the optimization required to determine the optimal/possible upgrade paths. For situations where no compatible solution can be reached from the start configuration, our proposal allows the user to interactively change the start configuration of the solution and apply a “what-if” scenario to find a configuration with the smallest number of incompatibilities.

The system 100 comprises a way to continuously monitor multi-device medical systems for incompatible connections, by representing the medical devices 120 as a connected graph 130 of nodes (devices) connected by (directed) edges, on which compatibility conditions are defined using product design requirements. These are used to (continuously) detect incompatibilities, addressing which may require local upgrades to nodes. The question of which upgrades to choose can be represented as a combinatorial optimization problem, and solved using (known) local search algorithms. Various potential solutions based on the preferred cost metric of the user (cost, time, functionality) are visualized for the user to choose the most valuable solution.

A network N of medical devices can be represented by a network N=(V, E) consisting of a set V of devices and a set E of connections. An example of a network N of medical devices is a patient monitoring network consisting of bed-side monitors, telemetry device, information centers, access points, storage servers, etc. A network can be dynamic in the sense that the set V may change over time and the connections in set E may change over time. The configuration of a network N=(V, E) at any given point of time is defined by a fixed status of all its devices and their mutual connections.

Each device v∈V is represented by a 3-tuple v=(t, hw, sw), where t represents the type of the device, hw represents the hardware revision of v, sw represents the software revision of v. t(v), hw(v) and sw(v) respectively represent the type, hardware revision and software revision of a given device v. There may be additional attributes that define the status of device v∈V. Vector A(v) denotes the complete ordered list of attributes that define the status of v, including t(v), hw(v) and sw(v).

Each connection e∈E can be assumed to be represent by a pair of devices (v, v′)∈V×V. The type t(e) of connection e indicates the type of connection, such as wired, wireless (802.11), wireless (Smart Hopping). Also, a connection e∈E may have additional attributes that describe the complete status of the connection. Vector A(e) denotes the complete ordered list of attributes that define the status of e, including t(e).

Not every pair of devices (v, v′) is mutually connectable. This depends, among others, on the types t(v) and t(v′) of the two devices. Let C(V)⊆V×V denote the set of pairs of connectable devices. In a given configuration of a system, not all connectable devices need be connected. For a connection between two connectable devices in a given configuration of the network, a binary value represents whether the potential connection exists in that configuration. This value is part of the full list of attributes for that connection, i.e., the vector A(e).

For a pair of connectable devices (v, v′), a connection e=(v, v′) is defined to be compatible if it satisfies the conditions that are defined on the attributes of the devices v and v′, notably their types and hardware and software revisions and the attributes of the connection, notably its type. The compatibility can be defined by a mapping c: A(v)×A(v′)×A(e)→{true, false} where c(e) is true if and only if the connection is compatible, i.e. all conditions are satisfied to guarantee a properly functioning connection.

Checking the compatibility of a given network configuration can be implemented by simply checking the compatibility of all individual connections. If a given network configuration is not compatible, then one or more connections must be incompatible.

The server computer 111 can include one or more modules to implement the disclosed operations of the method 200. A first module includes an “add new node” module. Using user input or automatic device detection/registration, this module maps an independent, functional (medical) device that is part of the overall medical solution to a node v∈V along with corresponding attributes A(v). A(v) includes the type t(v), hardware revision hw(v), and software revision sw(v) of v. A node can also represent other constraints/needs by the setup and/or customer which can then be respected by the search, e.g. certain requirements like the availability of a specific functionality/protocol/imaging sequence.

A second module includes an “add new connection” module. Using user input or automatic connection/dependency discovery, this module maps connections between devices to edges, along with corresponding attributes A(e). A(e) includes the type t(e) of e. For each edge, this module also stores the binary flag representing whether that edge exists in the current network configuration.

A third module includes a “network representation” module. Using input from the first and second modules, this module maintains the set of all nodes vᵢ∈V along with attributes A(v), the set of all edges eᵢ∈E along with attributes A(e), and the representation of the network of medical devices N=(V, E). Using user input, this module also maintains the set of connectable devices C(V)⊆V×V.

A fourth module includes an “edge compatibility” module. Using user input, this module stores the following compatibility condition for any edge e=(vᵢ, vⱼ) between any pair of connectable nodes (vᵢ, vⱼ)∈C(V) as maintained in the third module, as the value of the function

$$c : c : A ( {v}_{i} ) \times A ( {v}_{j} ) \times A ( e ) \to \{ True , False \}$$

A fifth module includes an “update edge compatibilities” module. Using user input—typically (updates to) product design requirements and product upgrade details, this module updates the value of the function c, i.e., the compatibility of the connection between each connectable device. Intuitively, changes to product design or upgrades may change attributes of devices or connections. This affects compatibility of connections, which is defined on such attributes. In some examples, combining user input at first setup time with continuous monitoring data over the medical system's lifetime (including field service and customer complaint data), the function c updates the value False for incompatible edges e to the actual probability of this incompatibility adversely affecting the medical system's functionality. Thus, d=0 instead of True for compatible nodes. Using user input via Module 3, d=1 for incompatible nodes at first setup time. Over the system's lifetime, d updates this value to the probability of impacted functionality by correlating (in time) known instances of the edge having been incompatible and the functionality having been affected. The former can be determined by, e.g., reading field service data and the latter by, e.g., reading customer complaint data. Thus, d is defined in this embodiment as the function: d: A(vᵢ)×A(vⱼ)×A(e)× [symbol] (N)× [symbol] (A(e), [symbol] (N)→[0,1]∈ [symbol] where [symbol] (N) is a function that generically represents the functionality of the medical system, and [symbol] is a function that correlates the times of the edge having had attributes A(e) with the above-mentioned functionality value of the medical system.

A sixth module includes a “network compatibility” module. This module iteratively invokes module 4 to (continuously) check if a given configuration of a network of medical devices N=(V, E) is compatible by checking whether the following holds A(vᵢ)×A(vⱼ)×A(e)→True, ∀(vᵢ, vⱼ)∈C(V). It stores all incompatible connections ê∈Ê for the configuration. In some examples, the overall compatibility of the network N is computed as either True or False at first setup time using user input. Over the system's lifetime, this embodiment of Module 6 uses the embodiment of Module 5 above to update this value to the overall probability of impacted probability as the product of the probabilities for individual edges.

A seventh module includes a “pre-upgrade network compatibility check” module. An “upgrade” can also refer to an “update” of a network configuration. This module is invoked (automatically) before any upgrade to any device or connection in the network of medical devices, or to the network configuration itself, or to the set of connectable devices C (V). Before the upgrade is actually applied, this module invokes the fifth module to import the future attributes A_{upgr}(v) and A_{upgr}(e) of each node v∈V_{upgr} and each edge e∈E_{upgr}, and the updated set of connectable devices C_{upgr}(V). Note that if the set of nodes (viz. edges) is not affected by an upgrade, then V_{upgr}=V (viz. E_{upgr}=E). Similarly, for nodes and edges not involved in the upgrade, A_{upgr}=A. Similarly, if the set of connectable devices does not change, then C_{upgr}(V)=C(V). Module 7 then invokes module 6 with the attributes A_{upgr} to verify whether compatibility holds for the network configuration that would result from the upgrade, by checking whether the following holds A_{upgr}(vᵢ)×A_{upgr}(vⱼ)×A_{upgr}(e)→True, ∀(vᵢ, vⱼ)∈C_{upgr}(V). If the above condition does not hold, i.e., if the result of the check is False, If it does, the upgrade progresses. In some examples, prior to any upgrade to any device or connection in the medical network, this module uses the sixth module to compute the probability of impacted medical functionality, with 0 corresponding to the value True in the original embodiment of this module. For any potential configuration with probability of impacted functionality>0, this module displays a warning to the user and asks for a confirmation of “Continue” or “Search alternative configuration.” If the former is chosen, the upgrade progresses.

An eighth module includes a “compatible network configuration search” module. This module applies a local search algorithm (e.g., simulated annealing) to find other alternative, compatible configurations Nₙₑₓₜ=(Vₙₑₓₜ, Eₙₑₓₜ) that can be reached from the current, incompatible configuration N_{upgr}=(V_{upgr}, E_{upgr}). For this, it uses the set of connectable devices that would result from the upgrade being considered C_{upgr} (V) and by invoking module 6 iteratively. It simulates local upgrades to each node vᵢ part of a connectable pair (vᵢ, vⱼ)∈C_{upgr}(V) to obtain the resulting attributes Aₙₑₓₜ(v), ∀ᵥ∈V_{upgr}. The set of nodes v∈V_{upgr} with attribute values Aₙₑₓₜ(v) instead of A_{upgr}(v) is then defined as the set Vₙₑₓₜ. Then, module 8 checks whether the following holds: Aₙₑₓₜ(vᵢ)×A_{upgr}(vⱼ)×A_{upgr}(e)→True, ∀(vᵢ, vⱼ)∈C_{upgr}(V). Each Aₙₑₓₜ(vᵢ) that satisfies the above condition replaces the older A_{upgr}(vᵢ) for that node vᵢ∈V_{upgr}. Thus Vₙₑₓₜ, the updated set of nodes is formed and used to create the alternative, compatible network configurations Nₙₑₓₜ=(Vₙₑₓₜ, Eₙₑₓₜ). For each such alternative configuration, this module also stores cumulative cost (if any) of the additional local upgrades to individual nodes in order to arrive at Nₙₑₓₜ=(Vₙₑₓₜ, Eₙₑₓₜ), cumulative time required by the additional local upgrades, and cumulative improvements to functionality of the overall medical system by summing the values of the corresponding attribute in the vector Aₙₑₓₜ(vᵢ) for each vᵢ∈Vₙₑₓₜ. This module can display these alternative configurations to the user along with the corresponding values computed above, and receives an input for a preferred configuration. The operation terminates. In some examples, when no alternative compatible network configurations can be found, this module displays the incompatible configuration provided by the seventh module that it began the search with to the user, along with a warning of incompatibility and asks for a confirmation of “Abort,” “Reset initial configuration”, or “Continue” (upon which the upgrade which would result in the incompatible network configuration progresses).

A ninth module includes a “network incompatibility fallback” module. This module iteratively prompts the user to modify the values of the attributes A(v) of each node v∈V in the original network configuration N=(V, E). It then invokes the seventh module to restart the pre-upgrade network compatibility check on this tweaked start configuration of the network.

### Second Example

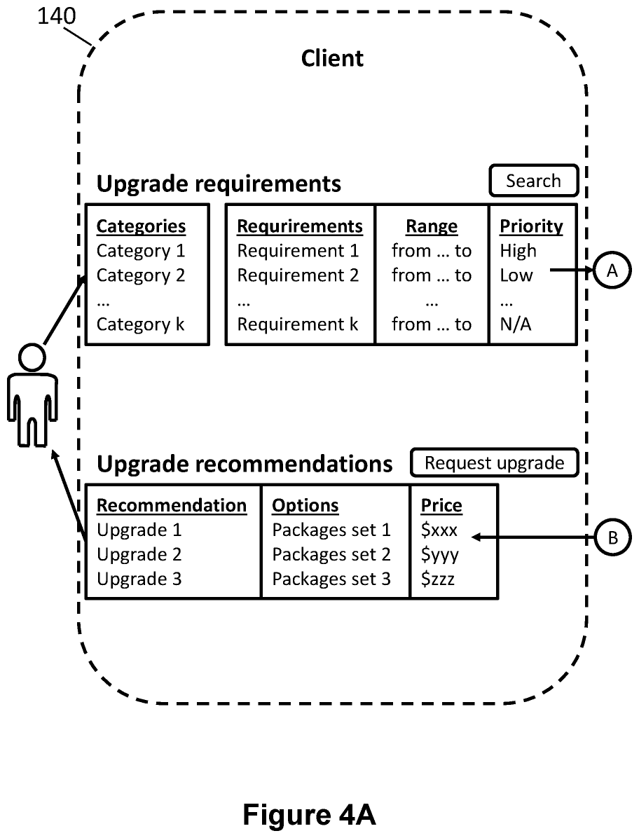

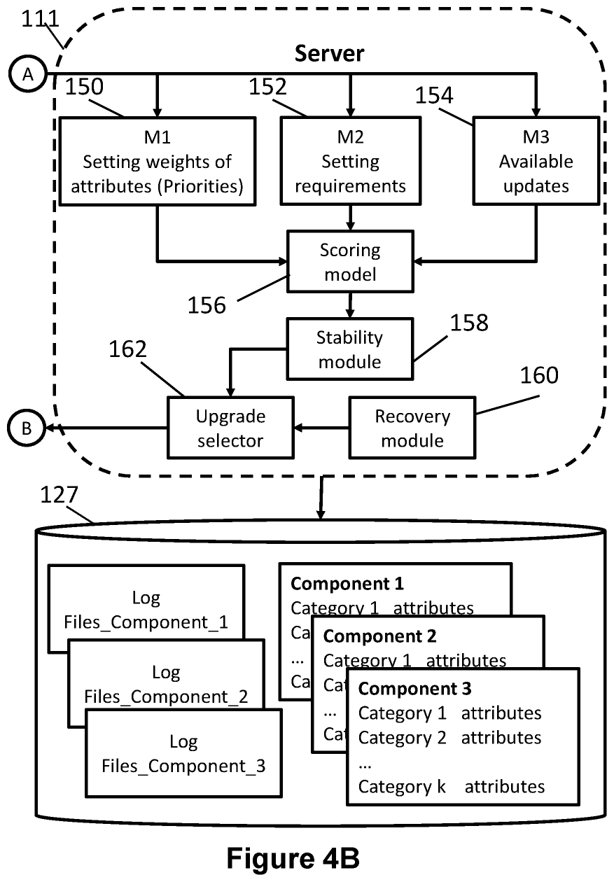

FIGS. 4A and 4B show an example of the system 100 implemented as a client server application. FIG. 4A diagrammatically illustrates the client side, while FIG. 4B diagrammatically illustrates the server side. A default set of m attributes for every manufacturer upgrade A={A₁, . . . , Aₘ} are defined, representing, for example, monetary cost, time required to apply, estimated downtime during the upgrade, resulting estimated uptime of the overall medical system post application, one or more main efficiency metrics such as sharpness of captured images (if the medical devices are medical imaging devices), resolution of the captured images, speed of operation of the medical devices, data communication latency of the medical devices (e.g., for medical devices such as patient monitors that wirelessly transmit data to the hospital server), and/or so forth. These default attributes can be defined by the manufacturer at the time of design.

A set of m weights W_{Ai} for and defined with each attribute Aᵢ. These weights determine the contribution of each attribute to the evaluated “score” of possible upgrade paths on the customer's needs.

A default set of n requirements or needs/constraints R={R₁, . . . , Rₙ} captured from the customer (hospital stakeholders) representing, for example, acceptable monetary cost of the upgrade, acceptable time required to apply, acceptable downtime of the medical system during the upgrade, expected resulting uptime of the system, expected value of the main efficiency metric relevant to the hospital. Note that these default requirements ideally correspond to the default attributes A of upgrades.

A set of n weights W_{Ci} for each category Cᵢ, where a category is a group of updates that are perceived by customers as interrelated and interchangeable (e.g., security updates, privacy updates, OS updates). These weights determine how much each requirement influences the final “score” of an upgrade path's attribute (by being evaluated against it).

A module M₁ 150 is configured to set the constraints R={R₁, . . . , Rₘ} for attributes A={A₁, . . . , Aₘ} weights W_{Ai} of the default attributes {A₁, . . . , Aₘ}. In one approach, the client side of FIG. 4A provides a user interface with check boxes, sliders, or other user interface dialogs via which a user can select the attributes to consider in scoring each potential upgrade (e.g., by checking or unchecking boxes next to attributes in a list of available attributes shown in the UI) and the weights to assign to the respective selected attributes (e.g., using sliders or the like associated with each checked attribute). These serve as the inputs to a module M₁ 150, whose output is an upgrade path with a vector of triples-attributes Ai, their values and their weights W_{Ai}. The client side user interface of FIG. 4A also enables the user to input or select the constraints, e.g. by selecting “from . . . to” ranges for corresponding constraints to specify, for example, an acceptable upgrade monetary cost range, an acceptable downtime range, acceptable value ranges for main efficiency metrics such as image sharpness, image resolution, medical device operating speed, data communication latency, and/or so forth. A module M₂ 152 is configured to set the priorities for selected categories based on these constraints received via the client side user interface of FIG. 4A. An output of the module M₂ 152 is a set of weights W_{Ci}. A module M₃ 154 is configured to create a list of available upgrades for selected categories. Operation of the module M₃ 154 depends on the upgrade categories selected via the client side, and may also depend on the user-specified constraints; e.g., a user-specified upper limit constraint on the acceptable monetary cost of the upgrade may rule out certain upgrades that would cost more than that upper limit. The output of the module M₃ 154 is a set of potential upgrades for scoring.

A scoring model 156 is configured to takes the output of the M₁, M₂, M₃ modules 150, 152, 154, and scores each potential upgrade and sorts based on score in order to deliver a sorted or ranked list of upgrade pathways. Each pathway in this way satisfies user requirements. A stability module 158 is configured to check compatibility of network configuration upgrades provided by the scoring model 156. This may for example employ the method of FIG. 2 to determine the compatibility status between the medical devices subject to be upgraded for each of the different potential upgrades identified by the module M₃ 154. An optional recovery module 160 is also configured to create a system checkpoint in case of upgrade failures. An upgrade selector module 162 is configured to show a user suitable upgrade pathways.

Referring back to FIG. 4A, from the client side the process begins when the user chooses set K of categories which they desire to upgrade, defines set of requirements (attributes or constraints) R, and specifies priorities on K. After pressing a “Search” button on the GUI 140, the client sends to the server 111 (FIG. 4B) a corresponding SQL request containing the list of categories, constraints and priorities. The server 111 initializes the module M₁ 150 and set weights to chosen categories according to priorities. The server 111 initializes module M₂ 152 and sets constraints according to the user request. The server 111 initializes module M₃ 154 and generates request to the non-transitory computer readable medium 127 for list of available upgrades in selected categories. Outputs of the module M₁ 150 (weights), module M₂ 152 (constraints), and module M₃ 154 (available relevant upgrades) are fed to the scoring model 156, which assigns a score to each potential upgrade identified by the module M₃ based on the compatibility status for that potential upgrade and the set of customer priorities and/or requirements. The scoring model 156 may, for example, use a linear optimization solver. The output of scoring model 156 is a sorted list of available upgrade paths and vertices (e.g., software versions) that meet user requirements. Each element from the list above is checked by the stability module 158 for compatibility of network configuration upgrades. The server 111 returns the list of checked pathways and vertices to the client, and the client shows it to the user via the GUI 140 of FIG. 4A as an indication 130 of at least the highest-scoring potential upgrade (e.g., in the nonlimiting illustrative example of FIG. 4A, listed “Upgrade 1”, “Upgrade 2”, and “Upgrade 3”). The user selects a suitable upgrade pathway and requests the upgrade by pressing the “Request upgrade” button shown in FIG. 4A.

This event of selecting an upgrade for implementation creates a ticket in a maintenance system, together with control point, and maintenance specialist provides a system update according to the selected pathway. In case of failure, the latest stable configuration system can be recovered using the recovery module 160. Alternatively, if the upgrade is a software upgrade that can be pushed automatically to the plurality of medical devices 120 via the Internet or another suitable electronic network, the user instruction to perform the upgrade can cause automatically push the software update to the plurality of medical devices 120 over the Internet or other electronic network.

In one example, modalities where any given single hospital has a very high number (in the order of hundreds or even thousands, e.g., in the case of multi-location hospitals) of medical systems, which are individually simple to upgrade. In such cases, the benefits in speed provided by the system 100 over a manual selection of the “best” possible upgrade path (and vertices) for all systems, allows it to be scalable and provide a winning edge and set of vertices (set of software versions). For example, in patient monitoring, large hospitals may contain a very high number of patient monitors. Individually, upgrading these systems is simple and fast. However, due to the high number, manually tailoring an upgrade path for all such systems (e.g., by an application specialist) is expensive. The system 100 automates that process and allows it to scale, reducing maintenance costs for the hospital.

In another example, modalities where any given hospital has a relatively small number (e.g., less than 50) of medical systems, which individually are complex to upgrade. For example, diagnostic imaging or interventional therapy systems. In such situations, the system 100 does not provide a clear benefit in speed over, e.g., an application specialist since the limited scope of systems makes it feasible and even efficient to manually tailor the upgrade path for the hospital. In such scenarios, the system 100 instead can provide recommendations to such a specialist on how the system software may be upgraded with minimal costs and maximal efficiency for a customer.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
data related to a plurality of medical devices; and
instructions readable and executable by at least one processor to:
determine, from the data related to the plurality of medical devices, a compatibility status between multiple medical devices of the plurality of medical devices; and
output, on a graphical user interface displayed on a display device, an indication of the compatibility status.

2. The non-transitory computer readable medium of claim 1, wherein the indication of the compatibility status comprises a compatible status or an incompatible status or comprises a probability of compatibility between multiple medical devices.

3. The non-transitory computer readable medium of claim 1, wherein the instructions are further executable by the at least one processor to:
determine a compatibility status of a potential software upgrade of the plurality of medical device by performing the determine and output operations for the plurality of medical devices with the potential upgrade to output, on the GUI, an upgrade compatibility status for the potential upgrade; and
receive, via the GUI, an instruction to perform the upgrade and in response automatically push the software upgrade to the plurality of medical devices over an electronic network.

4. The non-transitory computer readable medium of claim 1, wherein the data related to a plurality of medical devices comprises a representation including (i) the medical devices, (ii) connections therebetween, and (iii) formulations for checking for incompatibilities between the medical devices.

5. The non-transitory computer readable medium of claim 1, wherein the determination of the compatibility status includes:
analyzing potential updates for the plurality of medical devices; and
determining the compatibility status based on the analyzed potential updates.

6. The non-transitory computer readable medium of claim 5, wherein the potential updates comprise one or more potential hardware updates or potential software updates.

7. The non-transitory computer readable medium of claim 1, wherein the compatibility status between the multiple medical devices of the plurality of medical devices is a compatibility of an operative connection between a pair of medical devices of the plurality of medical devices.

8. The non-transitory computer readable medium, of claim 1, wherein:
the determination of the compatibility status between the multiple medical devices of the plurality of medical devices comprises generating a connected graph comprising nodes representing the medical devices of the plurality of medical devices and edges between pairs of nodes wherein each edge represents an operative connection between the medical devices represented by the nodes connected by that edge, and
the edges are labeled with compatibilities of the operative connections between the medical devices represented by the nodes connected by the respective edges.

9. The non-transitory computer readable medium of claim 8, wherein the instructions are further readable and executable by at least one processor to:
add one or more new edges to the connected graph, the determination of the compatibility status including determining compatibility statuses for the one or more new edges.

10. The non-transitory computer readable medium of claim 1, wherein the instructions are further readable and executable by at least one processor to:
add data related to a proposed new medical device to the data related to the plurality of medical devices, and the compatibility status is between the proposed new medical device and at least one other medical device of the plurality of medical devices with which the proposed new medical device is proposed to be connected.

11. The non-transitory computer readable medium of claim 1, wherein the instructions are further readable and executable by at least one processor to:
repeating the determination of the compatibility status between multiple medical devices of the plurality of medical devices for each of a plurality of different potential upgrades to the plurality of medical devices; and
assigning a score to each potential upgrade based on the compatibility status for that potential upgrade and a set of customer priorities and/or requirements including at least one of sharpness of images captured by the medical devices, resolution of images captured by the medical devices, speed of operation of the medical devices, and data communication latency of the medical devices;
wherein the output, on the GUI displayed on the display device, of the indication of the compatibility status includes outputting indications of the scores assigned to the respective potential upgrades.

12. The non-transitory computer readable medium of claim 11, wherein the instructions are further readable and executable by at least one processor to:
display, on the GUI, a user dialog via which the set of customer priorities and/or requirements is received.

13. A non-transitory computer readable medium storing:
data related to a plurality of medical devices and potential upgrades to the plurality of medical devices; and
instructions readable and executable by at least one processor to:
receive or determine a plurality of different potential upgrades for the plurality of medical devices;
provide a graphical user interface displayed on a display device via which a set of customer priorities and/or requirements are received;
assign a score to each potential upgrade based on the received set of customer priorities and/or requirements; and
output, on the GUI, an indication of at least the highest-scoring potential upgrade.

14. The non-transitory computer readable medium of claim 13, wherein the received set of customer priorities and/or requirements includes performance requirements for the medical devices including at least one of sharpness of images captured by the medical devices, resolution of images captured by the medical devices, speed of operation of the medical devices, and data communication latency of the medical devices.

15. The non-transitory computer readable medium of claim 13, wherein the instructions are further readable and executable by the at least one processor to:
receive, via the GUI, a selection of a potential software upgrade indicated by the output for implementation; and
in response to the selection, automatically push the software upgrade to the plurality of medical devices over an electronic network.

16. The non-transitory computer readable medium of claim 13, wherein the plurality of different potential upgrades for the plurality of medical devices are received via the GUI as what-if scenarios for upgrading the plurality of medical devices, and the performing of the what-if scenarios on the data related to a plurality of medical devices and potential upgrades to the plurality of medical devices includes:
analyzing one or more customer factors for the potential upgrades, the one or more customer factors including one or more of monetary cost, downtime, and compatibility of updated medical devices with non-updated medical devices;
scoring the one or more potential upgrades to the plurality of medical devices based on the analyzing of the one or more customer factors; and
selecting one or more potential upgrades based on the scoring.

17. The non-transitory computer readable medium of claim 13, wherein the instructions further include:
storing a status of each medical device prior to any implemented update; and
reverting at least one of the medical devices to the stored status responsive to a failed update.

18. A non-transitory computer readable medium storing:
data related to a plurality of medical devices; and
instructions readable and executable by at least one processor to:
determine, from the data related to the plurality of medical devices, a compatibility status between multiple medical devices of the plurality of medical devices, the compatibility status between the multiple medical devices of the plurality of medical devices is a compatibility of an operative connection between a pair of medical devices of the plurality of medical devices; and
output, on a graphical user interface (GUI) displayed on a display device, an indication of the compatibility status.

19. The non-transitory computer readable medium of claim 17, wherein:
the determination of the compatibility status between the multiple medical devices of the plurality of medical devices comprises generating a connected graph comprising nodes representing the medical devices of the plurality of medical devices and edges between pairs of nodes wherein each edge represents an operative connection between the medical devices represented by the nodes connected by that edge, and the edges are labeled with compatibilities of the operative connections between the medical devices represented by the nodes connected by the respective edges.

20. The non-transitory computer readable medium of claim 19, wherein the instructions are further readable and executable by at least one processor to:
add one or more new edges to the connected graph, the determination of the compatibility status including determining compatibility statuses for the one or more new edges.
